(19) United States
(12) Patent Application Publication
(10) Pub. No.: US 2006/0241133 A1
(43) Pub. Date: Oct. 26, 2006

(54) Electrically variable pneumatic structural element

(76) Inventors: Mark Steven Shearman, Bishops Stortford (GB); Mervyn Turner, Westfield, NJ (US)

Correspondence Address: MERCK AND CO., INC, P O BOX 2000, RAHWAY, NJ, 07065-0907, US

(21) Appl. No.: 10/549,839

(22) PCT Filed: Mar. 8, 2004
(86) PCT No.: PCT/GB04/00983

§ 371 Date: Jun. 22, 2006

(30) Foreign Application Priority Data
Mar. 14, 2003 (US) 60454589

(51) Int. Cl. A61K 31/4747

(52) U.S. Cl. 514/278

(57) ABSTRACT

There is disclosed the use of a compound of formula (I): or a pharmaceutically acceptable salt thereof, for the manufacture of a medicament for the treatment of age-related cognitive decline or mild cognitive impairment, in particular with a view to preventing or delaying the onset of Alzheimer's disease.

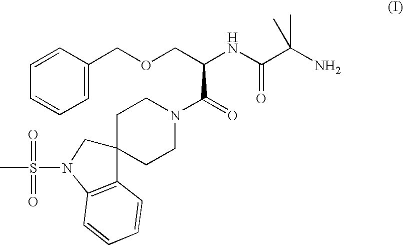

This invention relates to the use of methods and materials for therapeutic treatment of the human body. In particular, it provides methods of treating age related cognitive decline and mild cognitive impairment, especially in order to prevent or delay the onset of Alzheimer's disease.

Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). When used herein, age-related cognitive decline is characterized by a decline of at least six months' duration in at least one of: memory and learning; attention and concentration; thinking; language; and visuospatial functioning and a score of more than one standard deviation below the norm on standardized neuropsychologic testing such as the MMSE. In particular, there may be a progressive decline in memory. In the more severe condition mild cognitive impairment (MCI), the degree of memory impairment is outside the range considered normal for the age of the patient but Alzheimer's disease (AD) is not present. The differential diagnosis of MCI and mild AD is described by Petersen et al., Arch. Neurol., 56 (1999), 303-8. In the same article, Petersen et al. disclose that the patients suffering from MCI typically experience a progressive increase in cognitive impairment and in many cases develop AD. Further information on the differential diagnosis of MCI is provided by Knopman et al, Mayo Clinic Proceedings, 78 (2003), 1290-1308. In a study of elderly subjects, Tuokko et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Age-related cognitive decline and mild cognitive impairment are distinct from the significant cognitive deficit that sometimes results from cerebral or systemic diseases and traumas, such as stroke, concussion, or major disfunction of the pituitary.

Alzheimer's disease (AD) is the most prevalent form of dementia. Its diagnosis is described in the Diagnostic and Statistical Manual of Mental Disorders, 4^{th} ed., published by the American Psychiatric Association (DSM-IV) (e.g. pages 139-143). Diagnostic criteria for Alzheimer's disease includes the development of multiple cognitive deficits in a patient manifested by both (1) memory impairment (impaired ability to learn new information or to recall previously learned information), and (2) one (or more) of the following cognitive disturbances (a) aphasia (language disturbance), (b) apraxia (impaired ability to carry out motor activities despite intact motor function), (c) agnosia (failure to recognize or identify objects despite intact sensory function) and (d) disturbances in executing functioning (i.e. planning, organizing, sequencing, abstracting). Such cognitive deficits are not characterized as being due to any of the following: (1) other central nervous system conditions that cause progressive deficits in memory and cognition (e.g. cerebrovascular disease, Parkinson's disease, Huntington's disease, subdural hematoma, normal-pressure hydrocephalus, brain tumor), (2) systemic conditions that are known to cause dementia (e.g. hypothyroidism, vitamin B12 or folic acid deficiency, niacin deficiency, hypercalcemia, neurosyphilis, HIV infection), (3) substance-induced conditions.

Alzheimer's disease is a neurodegenerative disorder, clinically characterized by progressive loss of memory and general cognitive function, and pathologically characterized by the deposition of extracellular proteinaceous plaques in the cortical and associative brain regions of sufferers. These plaques mainly comprise fibrillar aggregates of β-amyloid peptide (Aβ). Reducing the burden of Aβ in the brain has therefore been proposed as a strategy for treatment of AD. For example Carro et al, in Nature Medicine, 8 (2002), 1390-7, disclose that subcutaneous administration of insulin-like growth factor 1 (IGF-1) causes a reduction in the cerebral Aβ burden in certain rodents. However, some authors have questioned whether secretion of Aβ is responsible for the neuronal loss that is generally held to be the immediate cause of dementia of Alzheimer's type (see, for example, Robinson and Bishop, Neurobiology of Aging, 23 (2002), 1051-72; and also New Scientist, Feb. 1, 2003, 35-37).

U.S. Pat. No. 5,767,124, U.S. Pat. No. 5,536,716, WO 94/13696 and EP 0615977B disclose compounds which are growth hormone secretagogues, i.e. which stimulate or increase the endogenous release of growth hormone in animals, including humans. This property is useful for promoting the growth of food animals, and in humans for treating physiological or medical conditions characterised by a deficiency in growth hormone secretion, and medical conditions which are improved by the anabolic effects of growth hormone. The listed conditions which can be treated include Alzheimer's Disease.

U.S. Pat. No. 4,902,680 advocates the administration of growth hormone to patients in the advanced stages of Alzheimer's disease.

WO 00/13650 discloses that increased levels of growth hormone in the brain provide a neuroprotective effect, and in particular can rescue neurons that would otherwise die as a result of an insult such as that associated with a neurodegenerative disease. The injection of growth hormone into the brain is contemplated.

Major growth hormone deficiency as a result of major pituitary dysfunction can lead to cognitive impairment in adults (Deijen et al., Psychoneuroendocrinology, 21 (1996), 313-22) which is reversible by growth hormone replacement therapy (Deijen et al., ibid., 23 (1998), 45-55; Soares et al., Arq. Neuropsiquiatr., 57 (1999), 182-9; Rosen et al., Horm. Res., 43 (1995), 93-99). It has also been speculated that administration of growth hormone or a secretagogue thereof might, amongst many other effects, improve cognition in normal healthy elderly subjects (see, for example, Merriam et al., Endocrine, 7 (1997), 49-52; Cummings and Merriam, Semin. Reprod. Endocrinol., 17 (1999), 311-25) but no actual use was made of the secretagogues for such a purpose. However, recent publications conclude that the administration of growth hormone or growth hormone secretagogues to normal ageing subjects is not of clinical benefit (Anawalt and Merriam, Endocrinol. Metab. Clin. North Am., 30 (2001), 647-69; Cummings and Merriam, Annu. Rev. Med., 54 (2003), 513-33). This invention is not concerned with normal healthy subjects (i.e. asymptomatic subjects).

A compound disclosed in the aforementioned U.S. Pat. No. 5,767,124 has been the subject of a number of clinical trials in various therapeutic fields but not age-related cognitive decline or mild cognitive impairment (see, for example, Murphy et al, J. Bone Miner. Res., 14, (1999), 1182-8; Chapman et al, J. Clinical Endocrinology and Metabolism, 81, (1996), 4249-57; ibid., 82, (1997), 3455-63; and Svensson et al, ibid., 83, (1998), 362-9).

According to the invention, there is provided the use of a compound of formula I: 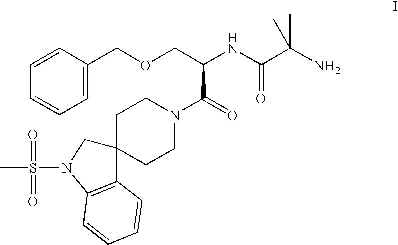
or a pharmaceutically acceptable salt thereof,
for the manufacture of a medicament for the treatment of age-related cognitive decline or mild cognitive impairment. A favoured outcome of the treatment is prevention or delay of the onset of Alzheimer's disease. Such prevention or delay may be evidenced by halting or slowing of the patient's cognitive decline, or halting or slowing of the progress of cognitive impairment.

It is one of the benefits of this invention that said medicament need not be administrable by injection. In a preferred embodiment, said medicament is in a form suitable for oral administration.

The compound of formula I may be termed N-[1(R)-[(1,2-dihydro-1-methanesulfonylspiro[3H-indole-3,4′-piperidin]-1′-yl)carbonyl]-2-(phenylmethyloxy)ethyl]-2-amino-2-methylpropanamide.

For use in the invention, the compound of formula I is advantageously in the form of an acid addition salt formed by interaction of the primary amine group in I with a pharmaceutically acceptable acid such as hydrochloric acid, sulphuric acid, benzenesulfonic acid, toluenesulfonic acid, methanesulfonic acid, fumaric acid, maleic acid, succinic acid, acetic acid, benzoic acid, oxalic acid, citric acid, tartaric acid, carbonic acid or phosphoric acid. Preferably, the compound of formula I is in the form of the methanesulfonate salt, which is itself preferably in one of the polymorphic forms described in U.S. Pat. No. 5,767,124.

The synthesis of the compound of formula I and suitable salts thereof is described in U.S. Pat. No. 5,767,124, EP 0615977B, U.S. Pat. No. 6,028,196, U.S. Pat. No. 6,046,333 and WO 96/33189.

The method of this invention may lead to enhanced clearance of Aβ from the brain, which may be evidenced by altered levels of soluble Aβ in the cerebrospinal fluid and/or serum. Alternatively (or additionally), imaging techniques such as magnetic resonance imaging, positron emission tomography, single photon emission computed tomography and multiphoton microscopy may be employed to monitor the extent of AD deposition in the brain (see, for example, Bacskai et al., J. Cereb. Blood Flow Metab., 22 (2002), 1035-41).

In a particular embodiment, the invention provides the use of a compound as defined above for the manufacture of a medicament for preventing or delaying the onset of dementia associated with Alzheimer's disease in a patient with age related cognitive decline or in a patient with mild cognitive impairment.

The invention also provides a method of treating age-related cognitive decline or mild cognitive impairment comprising administering to a patient in need thereof a therapeutically-effective amount of a compound of formula I as defined above or a pharmaceutically acceptable In a particular embodiment, the invention also provides a method of preventing, retarding or arresting any further age-related cognitive decline or progression of mild cognitive impairment comprising administering to a patient in need thereof a therapeutically-effective amount of a compound of formula I as defined above or a pharmaceutically acceptable salt thereof.

The invention also provides a method of preventing or delaying the onset of Alzheimer's disease comprising administering to a patient suffering from age related cognitive decline or mild cognitive impairment a therapeutically-effective amount of a compound of formula I as defined above or a pharmaceutically acceptable salt thereof.

In a particular embodiment, the invention also provides a method of preventing or delaying the onset of dementia associated with Alzheimer's disease comprising administering to a patient suffering from age-related cognitive decline or mild cognitive impairment a therapeutically-effective amount of a compound of formula I as defined above or a pharmaceutically acceptable salt thereof.

In a favoured embodiment of this invention, the compound of formula I or a pharmaceutically acceptable salt thereof is administered to a person having mild cognitive impairment.

In another favoured embodiment of this invention, the compound of formula I or a pharmaceutically acceptable salt thereof is administered to a person exhibiting age-related cognitive decline.

The methods of this invention are most apt for preventing, retarding or arresting the accumulation of insoluble Aβ in the brain of a patient suffering from age-related cognitive decline or mild cognitive impairment.

The medicaments useful in the invention are particularly suitable for administration to patients who suffer impaired memory function but do not exhibit other symptoms that would constitute dementia, such as aphasia, apraxia, agnosia or disturbance in executive functioning. Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; adult-onset diabetes mellitus; raised CSF levels of total tau; raised CSF levels of phospho-tau; and lowered CSF levels of Aβ42.

In a particular embodiment of the invention, the compound of formula I or a pharmaceutically acceptable salt thereof is administered to a patient suffering from age-related cognitive decline or mild cognitive impairment who additionally possesses one or more risk factors for developing AD selected from: a family history of the disease; a genetic predisposition to the disease; elevated serum cholesterol; adult-onset diabetes mellitus; raised CSF levels of total tau; raised CSF levels of phospho-tau; and lowered CSF levels of Aβ42.

A genetic predisposition (especially towards early onset Alzheimer's disease) can arise from point mutations in one or more of the APP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment with the compound of formula I or a pharmaceutically acceptable salt thereof, so that changes therein may be detected, e.g. the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med., 12 (1982), 397408; Cockrell et al., Psychopharmacology, 24 (1988), 689-692; Crum et al., J. Am. Med. Assoc'n. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment.

The compounds suitable for use in the invention are typically administered as pharmaceutical compositions comprising the compound of formula I (or pharmaceutically acceptable salt thereof) and a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and polyethylene glycol, and other pharmaceutical diluents, e.g. water, to form a homogeneous preformulation composition containing a compound of formula I or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This preformulation composition is then subdivided into unit dosage forms of the type described above, e.g. containing from 0.1 to about 150 mg of the active ingredient. Favoured unit dosage forms contain from 0.5 to 100 mg, for example 0.5, 1, 2, 3, 5, 10, 15, 20, 25, 30, 50, 60 or 75 mg, of the free base or equivalent quantities of a pharmaceutically acceptable salt thereof. Tablets or pills of the pharmaceutical composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the pharmaceutical compositions may be incorporated for administration orally include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) or gelatin.

Pharmaceutical formulations of the compound of formula I and suitable salts thereof are described in U.S. Pat. No. 6,123,964.

For use in the invention, the optimum dosage level of the compounds defined above, in terms of safety and efficacy, may vary according to the perceived risk of developing Alzheimer's disease, and/or other factors specific to the individual patient, and may be determined by methods well known to those skilled in the art. Generally speaking, doses of about 0.01 to 5.0 mg/kg per day, preferably about 0.05 to 2.5 mg/kg per day, more preferably about 0.1 to 1.0 mg/kg of body weight per day, may be contemplated.

The compounds may be administered on any suitable regimen, for example 1, 2, 3 or 4 times per day, but administration once or twice per day is preferred, with administration once per day most preferred. Said administration is preferably oral.

The compounds may be administered at regular intervals over an extended period, e.g. of 3 months, 6 months, 1 year, or more, or for the remaining lifetime of the subject.

The compound of formula I and its pharmaceutically acceptable salts are particularly suitable for oral administration. Hence, orally administrable unit dose pharmaceutical compositions are most aptly employed in the methods and uses of this invention.

In one embodiment of the invention, the compound N-[1(R)-[(1,2-dihydro-1-methanesulfonylspiro[3H-indole-3,4′-piperidin]-1′-yl)carbonyl]-2-(phenylmethyloxy)ethyl]-2-amino-2-methylpropanamide methanesulfonate is administered at a total dose equivalent to about 25 mg of the free base per person per day. In two further embodiments said compound is administered in a total dose equivalent to 10 mg or 5 mg of free base per day. In these embodiments it is preferred that the daily dose is administered at a single time, for example as one tablet or two tablets.

Other regimens and/or dosage levels outside the limits outlined above may be used if circumstances so demand.

### EXAMPLES

In the following examples, the active drug is N-[1(R)-[(1,2-dihydro-1-methanesulfonylspiro[3H-indole-3,4′-piperidin]-1′-yl)carbonyl]-2-(phenylmethyloxy)ethyl]-2-amino-2-methylpropanamide methanesulfonate, prepared in crystalline form I as described in U.S. Pat. No. 5,767,124.

### Example 1

### Tablet Formulation (Wet Granulation Method)

The following formulation is used to manufacture coated tablets, each containing the equivalent of 25 mg free base:

| | mg/tablet |
|---|---|
| Tablet Core | |
| active drug | 29.6 |
| Starch Pregelatinized NF | 113.0 |
| Calcium Phosphate Dibasic, USP | 174.0 |
| Microcrystalline Cellulose NF | 57.0 |
| Magnesium Stearate Impalpable Powder NF | 2.0 |
| Croscarmellose Sodium NF | 24.0 |
| Ethanol 95% USP* | — |
| Water Purified USP* | — |
| Tablet Coat | |
| Hydroxypropyl Methylcellulose USP | 3.2 |
| Hydroxypropyl Cellulose w/ 0.3% Silica | 3.2 |
| Titanium Dioxide USP | 1.28 |
| Talc USP Purified | 0.32 |
| Water Purified USP* | — |

*removed during process.

The active drug, calcium phosphate, starch pregelatinized, microcrystalline cellulose and half of the croscarmellose sodium are mixed and then transferred to a granulator for brief additional mixing. A 25% ethanol/water mixture is added slowly and granulation performed. The granules are dried, screened, and mixed with the remaining croscarmellose sodium and then with the magnesium stearate. Tablets are formed by compression and film coated by spraying with an aqueous suspension of the tablet coat ingredients.

Tablets containing different loadings of the drug, for example 10 mg or 5 mg, are formed by the same procedure, with appropriate adjustment of the relative proportions of the ingredients.

### Example 2

### Tablet Formulation (Roller Compaction Process)

The following formulation is used to manufacture coated tablets, each containing the equivalent of 25 mg free base:

| | mg/tablet |
|---|---|
| Tablet Core | |
| active drug | 29.6 |
| Calcium Phosphate Dibasic, USP | 60.44 |
| Microcrystalline Cellulose NF | 150.0 |
| Magnesium Stearate Impalpable Powder NF | 2.56 |
| Croscarmellose Sodium NF | 7.5 |
| Tablet Coat | |
| Hydroxypropyl Methylcellulose USP | 3.125 |
| Hydroxypropyl Cellulose w/ 0.3% Silica | 3.125 |
| Titanium Dioxide USP | 1.25 |
| Water Purified USP* | — |

*removed during process.

The active drug, microcrystalline cellulose and a portion of the magnesium stearate are mixed, then roller compacted, and the resulting compact is milled. The milled material is blended with the croscarmellose sodium, calcium phosphate and the remaining magnesium stearate, then compressed into tablets. The tablets are film coated by spraying with an aqueous suspension of the tablet coat ingredients.

Tablets containing different loadings of the drug are formed by the same procedure, with appropriate adjustment of the relative proportions of the ingredients.

### Example 3

### Treatment for Preventing or Delaying the Onset of Alzheimer's Disease

One 25 mg tablet (as described in Example 1 or Example 2) is administered daily with water to subjects in need of such treatment.

### Example 4

### Treatment for Preventing or Delaying the Onset of Alzheimer's Disease in a Subject Exhibiting Mild Cognitive Impairment

A subject having mild cognitive impairment is identified using the MMSE or similar diagnostic tool.

One 25 mg tablet (as described in Example 1 or Example 2) is administered daily with water to said subject.

The cognitive status of the subject is monitored periodically using the MMSE or similar tool, and the subject is monitored for clinical symptoms of dementia.

1-11. (canceled)

12. A method for treating age-related cognitive decline or mild cognitive impairment in a human patient in need thereof comprising administering to the patient a therapeutically effective amount of the compound of formula I: 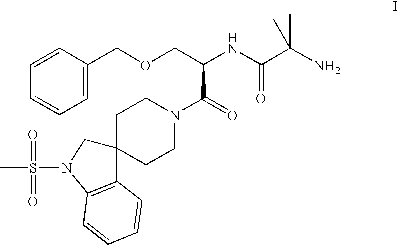
or a pharmaceutically acceptable salt thereof.

13. The method of claim 12 wherein the patient is a person aged 55 or over.

14. The method of claim 13 wherein the patient is a person aged 60 or over.

15. The method of claim 14 wherein the patient is a person aged 65 or over.

16. The method of claim 12 wherein the patient possesses one or more risk factors for developing Alzheimer's disease selected from: a family history of Alzheimer's disease; a genetic predisposition to Alzheimer's disease; elevated serum cholesterol; adult-onset diabetes mellitus; raised cerebro-spinal fluid levels of total tau; raised cerebro-spinal fluid levels of phospho-tau; and lowered cerebro-spinal fluid levels of Aβ42.

17. The method of claim 12 wherein the patient suffers from mild cognitive impairment.

18. The method of claim 12 wherein the patient suffers from age-related cognitive decline.

19. The method of claim 12 wherein the treatment of age-related cognitive decline or mild cognitive impairment in a human patient comprises preventing, retarding or arresting the accumulation of insoluble Aβ in the brain of the patient suffering from age-related cognitive decline or mild cognitive impairment.

20. A method for preventing or delaying the onset of Alzheimer's disease in a human patient suffering from age-related cognitive decline or mild cognitive impairment comprising administering to the patient a therapeutically effective amount of the compound of formula I: 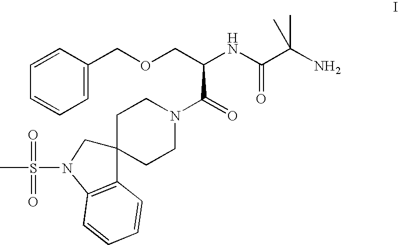
or a pharmaceutically acceptable salt thereof.

21. The method of claim 20 wherein the patient is a person aged 55 or over.

22. The method of claim 21 wherein the patient is a person aged 60 or over.

23. The method of claim 22 wherein the patient is a person aged 65 or over.

24. The method of claim 20 wherein the patient additionally possesses one or more risk factors for developing Alzheimer's disease selected from: a family history of Alzheimer's disease; a genetic predisposition to Alzheimer's disease; elevated serum cholesterol; adult-onset diabetes mellitus; raised cerebro-spinal fluid levels of total tau; raised cerebro-spinal fluid levels of phospho-tau; and lowered cerebro-spinal fluid levels of Aβ42.

25. The method of claim 20 wherein the patient suffers from mild cognitive impairment.

26. The method of claim 20 wherein the patient suffers from age-related cognitive decline.

27. A method for preventing or delaying the onset of dementia associated with Alzheimer's disease in a patient suffering from age-related cognitive decline or mild cognitive impairment comprising administering to the patient a therapeutically effective amount of the compound of formula I: 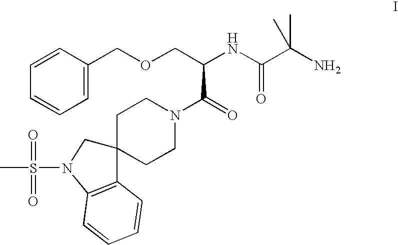
or a pharmaceutically acceptable salt thereof.

28. The method of claim 27 wherein the patient is a person aged 55 or over.

29. The method of claim 28 wherein the patient is a person aged 60 or over.

30. The method of claim 29 wherein the patient is a person aged 65 or over.

31. The method of claim 27 wherein the patient additionally possesses one or more risk factors for developing Alzheimer's disease selected from: a family history of Alzheimer's disease; a genetic predisposition to Alzheimer's disease; elevated serum cholesterol; adult-onset diabetes mellitus; raised cerebro-spinal fluid levels of total tau; raised cerebro-spinal fluid levels of phospho-tau; and lowered cerebro-spinal fluid levels of Aβ42.

32. The method of claim 27 wherein the patient suffers from mild cognitive impairment.

33. The method of claim 27 wherein the patient suffers from age-related cognitive decline.
